(19) United States
(12) Patent Application Publication
(10) Pub. No.: US 2016/0082110 A1
(43) Pub. Date: Mar. 24, 2016

(54) BIOACTIVE TERPOLYMER COMPOSITIONS AND METHODS OF MAKING AND USING SAME

(71) Applicant: SurModics, Inc., Eden Prairie, MN (US)

(72) Inventors: Kevin W. Burton, Hoover, AL (US); Howard Bowman, Birmingham, AL (US); Danielle Biggs, Memphis, TN (US); Peter Markland, Birmingham, AL (US)

(21) Appl. No.: 14/955,528

(22) Filed: Dec. 1, 2015

Related U.S. Application Data
(63) Continuation of application No. 12/644,097, filed on Dec. 22, 2009.
(60) Provisional application No. 61/140,476, filed on Dec. 23, 2008.

(51) Int. Cl. A61K 47/34; A61M 5/19; A61K 9/00

(52) U.S. Cl. CPC A61K 47/34; A61K 9/0019; A61M 5/19

(57) ABSTRACT

Described herein are terpolymer compositions, kits comprising the compositions, implant devices comprising the compositions, and methods of making and using same, including point of use methods.

### CROSS-REFERENCE TO RELATED APPLICATIONS

This application is based upon and claims the benefit of priority from prior U.S. Provisional Application Numbers 61/140,476, filed Dec. 23, 2008, the entire contents of which are incorporated herein by reference.

### BACKGROUND

In medicine, certain disorders and conditions require medical implants. Medical implants are often used to replace a damaged biological tissue or fluid, augment or enhance a biological process, enhance the healing of a surgical site, deliver a drug to a localized site within a subject, or perform another biological or structural role. Implants can even be necessary to keep a patient alive. Unfortunately, problems can arise during an implant surgery, or after a patient has received the medical implant. In some instances, the implant can impair healing of the surgical site. For example, the surface of the implant can recruit cellular debris and other biological material that can become infected with bacteria, fungus, or other infectious agents. The subject's immune system can also recognize the implant as a foreign body, and attempt to fight the implant using natural defenses. This often lowers the strength of the subject's immune system and can lead to further serious problems, such as periprosthetic infections, or other infections at or near the surgical implant site.

Accordingly, it can also be desirable to deliver a bioactive agent at or near the tissue adjacent the implant site. Such a bioactive agent can help prevent at least some of the aforementioned problems associated with implants, or enhance the function of the implant itself. Unfortunately, configuring each implant to be capable of locally delivering a bioactive agent is not always possible or practical. For example, regulations for the manufacture of drug products differ significantly from the regulations for the manufacture of medical devices.

As such, a need exists for compositions that can be applied to an implant device or near an implant site in a subject that effectively provide a bioactive agent at or near tissue adjacent the implant site. These needs and other needs are satisfied by the present invention.

### SUMMARY

Described herein are compositions comprising a terpolymer and a bioactive agent, kits comprising the terpolymers, bioactive agents, and compositions, and implant devices comprising the terpolymers, bioactive agents, and compositions. In one aspect, disclosed are point of use applications, wherein a bioactive agent is applied to a medical device close to the time of use, which allows for the separate and more rapid development of the bioactive agent and the implant device, such that the quality or efficacy of the final implant device is not unduly compromised.

In one aspect, disclosed is a bioactive agent delivery composition comprising: an adhering biocompatible terpolymer and a releasable bioactive agent.

In another aspect, disclosed is kit comprising an adhering biocompatible terpolymer and a bioactive agent.

In another aspect, disclosed is an implant device having a first implant device surface comprising the a composition comprising an adhering biocompatible terpolymer and a releasable bioactive agent, wherein the composition is present of any of claims 1-7 on at least a portion of the first implant device surface.

Also disclosed are methods of applying a coating to an implant device, the method comprising applying a coating comprising the 1) adhering biocompatible terpolymer and 2) the bioactive agent of a disclosed kit onto a surface of the implant device.

Also disclosed are methods for formulating a bioactive composition, the method comprising mixing an adhering biocompatible terpolymer and a bioactive agent, thereby forming the bioactive composition.

The advantages of the invention will be set forth in part in the description which follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### DETAILED DESCRIPTION

Before the present compounds, compositions, compositions, articles, devices and/or methods are disclosed and described, it is to be understood that the aspects described below are not limited to specific compounds, compositions, compositions, articles, devices, methods, or uses as such may, of course, vary. It is also to be understood that the terminology used herein is for the purpose of describing particular aspects only and is not intended to be limiting.

In this specification and in the claims that follow, reference will be made to a number of terms that shall be defined to have the following meanings:

Throughout this specification, unless the context requires otherwise, the word “comprise,” or variations such as “comprises” or “comprising,” will be understood to imply the inclusion of a stated integer or step or group of integers or steps but not the exclusion of any other integer or step or group of integers or steps.

It must be noted that, as used in the specification and the appended claims, the singular forms “a,” “an” and “the” include plural referents unless the context clearly dictates otherwise. Thus, for example, reference to “a bioactive agent” includes mixtures of two or more such agents, and the like.

“Optional” or “optionally” means that the subsequently described event or circumstance can or cannot occur, and that the description includes instances where the event or circumstance occurs and instances where it does not.

Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

A weight percent of a component, unless specifically stated to the contrary, is based on the total weight of the formulation or composition in which the component is included.

The term “adhering terpolymer” refers to a terpolymer that can adhere to a contacting surface. The “adhering terpolymer” can be any terpolymer capable of sticking to a surface for a desired time period. In one aspect, an “adhering terpolymer” can act like, or be, an adhesive, a gel, a wax or waxy polymer, a Vaseline® like material, a viscous terpolymer, or a tacky terpolymer.

The term “biocompatible” refers a substance that is substantially non-toxic to a subject. “Biodegradable” is generally referred to herein as a material that will erode to soluble species or that will degrade under physiologic conditions to smaller units or chemical species that are, themselves, non-toxic (biocompatible) to the subject and capable of being metabolized, eliminated, or excreted by the subject.

A “bioactive agent” refers to an agent that has biological activity. The biological agent can be used to treat, diagnose, cure, mitigate, prevent (i.e., prophylactically), ameliorate, modulate, or have an otherwise favorable effect on a disease, disorder, infection, and the like. A “releasable bioactive agent” is one that can be released from a disclosed polymer matrix. Bioactive agents also include those substances which affect the structure or function of a subject, or a pro-drug, which becomes bioactive or more bioactive after it has been placed in a predetermined physiological environment.

Disclosed are compounds, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a number of different polymers and agents are disclosed and discussed, each and every combination and permutation of the polymer and agent are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this disclosure including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

Described herein are compositions that can be applied to an implant device, or to a tissue or fluid of a subject. The compositions can release a bioactive agent into the subject. The compositions described herein allow for controlled-release, extended-release, modified-release, sustained-release, pulsatile-release, delayed-release, or programmed-release of the bioactive agent.

The terpolymer used with the compositions can be any suitable biocompatible terpolymer. In one aspect, the terpolymer is a biodegradable or non-biodegradable terpolymer. The polymers can be block or blocky ter-polymers, random ter-polymers, or star terpolymers. Any desired molecular weight terpolymer can be used, depending on the desired properties of the composition. In certain aspects, if a high strength composition is desired, then high molecular weight terpolymers can be used, for example, to meet strength requirements. In other aspects, low or medium molecular weight terpolymers can be used when, for example, when resorption time of the terpolymer, rather than material strength is desired.

The molecular weight of the terpolymer can be selected so as to provide a desired property of the composition. In certain aspects, the terpolymer can be provided by forming an adhering formulation of the terpolymer. In such aspects, the molecular weight should be high enough, or low enough, so that it forms satisfactory formulations. The molecular weight of a terpolymer is also important from the point of view that molecular weight influences the biodegradation rate of the terpolymer. For a diffusional mechanism of bioactive agent release, the terpolymer should remain intact until all of the drug is released from the terpolymer and then degrade. The drug can also be released from the terpolymer as the terpolymer bioerodes. By an appropriate selection of polymeric materials, a terpolymer formulation can be made such that the resulting biodegradable terpolymer exhibits both diffusional release and biodegradation release properties. Molecular weights can be measured by methods known in the art, including gel permeation chromatography, viscosity, light-scattering, among other methods.

The biodegrable terpolymer can be formulated so as to degrade within a desired time interval, once present in a subject. In some aspects, the time interval can be from about less than one day to about 1 month. Longer time intervals can extend to 6 months, including for example, terpolymer matrices that degrade from about ≧0 to about 6 months, or from about 1 to about 6 months. In other aspects, the terpolymer can degrade in longer time intervals, up to 2 years or longer, including, for example, from about ≧0 to about 2 years, or from about 1 month to about 2 years.

The desired bioactive agent release mechanism can influence the selection of the terpolymer. A composition can be selected so as to release or allow the release of a bioactive agent therefrom at a desired lapsed time after the composition has been implanted into a subject. In one aspect, the composition or terpolymer can be selected to release or allow the release of the bioactive agent prior to the bioactive agent beginning to diminish its activity, as the bioactive agent begins to diminish in activity, when the bioactive agent is partially diminished in activity, for example at least 25%, at least 50% or at least 75% diminished, when the bioactive agent is substantially diminished in activity, or when the bioactive agent is completely gone or no longer has activity.

In one aspect, the terpolymer can be a terpolymer that comprises one or more of polyesters, polyhydroxyalkanoates, polyhydroxybutyrates, polydioxanones, polyhydroxyvalerates, polyanhydrides, polyorthoesters, polyphosphazenes, polyphosphates, polyphosphoesters, polydioxanones, polyphosphoesters, polyphosphates, polyphosphonates, polyphosphates, polyhydroxyalkanoates, polycarbonates, polyalkylcarbonates, polyorthocarbonates, polyesteramides, polyamides, polyamines, polypeptides, polyurethanes, polyalkylene alkylates, polyalkylene oxalates, polyalkylene succinates, polyhydroxy fatty acids, polyacetals, polycyanoacrylates, polyketals, polyetheresters, polyethers, polyalkylene glycols, polyalkylene oxides, polyethylene glycols, polyethylene oxides, polypeptides, polysaccharides, or polyvinyl pyrrolidones. Other non-biodegradable but durable polymers include without limitation ethylene-vinyl acetate co-polymer, polytetrafluoroethylene, polypropylene, polyethylene, and the like. Likewise, other suitable non-biodegradable terpolymers include without limitation those that comprise silicones and polyurethanes.

In a further aspect, the terpolymer can be a terpolymer that comprises a poly(lactide), a poly(glycolide), a poly(lactide-co-glycolide), a poly(caprolactone), a poly(orthoester), a poly(phosphazene), a poly(hydroxybutyrate) or a copolymer containing a poly(hydroxybutarate), a poly(lactide-co-caprolactone), a polycarbonate, a polyesteramide, a polyanhydride, a poly(dioxanone), a poly(alkylene alkylate), a copolymer of polyethylene glycol and a polyorthoester, a biodegradable polyurethane, a poly(amino acid), a polyamide, a polyesteramide, a polyetherester, a polyacetal, a polycyanoacrylate, a poly(oxyethylene)/poly(oxypropylene) copolymer, polyacetals, polyketals, polyphosphoesters, polyhydroxyvalerates or a copolymer containing a polyhydroxyvalerate, polyalkylene oxalates, polyalkylene succinates, poly(maleic acid), and combinations, or blends thereof.

In a still further aspect, useful biodegradable terpolymers are those that comprise one or more residues of lactic acid, glycolic acid, lactide, glycolide, caprolactone, hydroxybutyrate, hydroxyvalerates, dioxanones, polyethylene glycol (PEG), polyethylene oxide, or a combination thereof. In a still further aspect, useful biodegradable terpolymers are those that comprise one or more residues of lactide, glycolide, caprolactone, or a combination thereof.

In one aspect, useful biodegradable terpolymers are those that comprise one or more blocks of hydrophilic or water soluble polymers, including, but not limited to, polyethylene glycol, (PEG), or polyvinyl pyrrolidone (PVP), in combination with one or more blocks another biocompabible or biodegradable terpolymer that comprises lactide, glycolide, caprolactone, or a combination thereof.

In specific aspects, the biodegradable terpolymer can comprise one or more lactide residues. To that end, the terpolymer can comprise any lactide residue, including all racemic and stereospecific forms of lactide, including, but not limited to, L-lactide, D-lactide, and D,L-lactide, or a mixture thereof. Useful terpolymers comprising lactide include, but are not limited to terpolymers comprising poly(L-lactide), poly(D-lactide), and poly(DL-lactide); and poly(lactide-co-glycolide), including poly(L-lactide-co-glycolide), poly(D-lactide-co-glycolide), and poly(DL-lactide-co-glycolide); or copolymers, terpolymers, combinations, or blends thereof. Lactide/glycolide terpolymers can be conveniently made by melt polymerization through ring opening of lactide and glycolide monomers. Additionally, racemic DL-lactide, L-lactide, and D-lactide polymers are commercially available. The L-polymers are more crystalline and resorb slower than DL-polymers. In addition to terpolymers comprising glycolide and DL-lactide or L-lactide, copolymers of L-lactide and DL-lactide are commercially available. Homopolymers of lactide or glycolide are also commercially available. These polymers can be combined to form a terpolymer

When the biodegradable terpolymer is terpolymer comprising poly(lactide-co-glycolide), poly(lactide), or poly(glycolide), the amount of lactide and glycolide in the terpolymer can vary. In a further aspect, the biodegradable terpolymer contains 0 to 100 mole %, 40 to 100 mole %, 50 to 100 mole %, 60 to 100 mole %, 70 to 100 mole %, or 80 to 100 mole % lactide and from 0 to 100 mole %, 0 to 60 mole %, 10 to 40 mole %, 20 to 40 mole %, or 30 to 40 mole % glycolide, wherein the amount of lactide and glycolide is 100 mole %. In a further aspect, the biodegradable terpolymer can be a terpolymer comprisnig poly(lactide), 95:5 poly(lactide-co-glycolide) 85:15 poly(lactide-co-glycolide), 75:25 poly(lactide-co-glycolide), 65:35 poly(lactide-co-glycolide), or 50:50 poly(lactide-co-glycolide), where the ratios are mole ratios.

In a further aspect, the terpolymer can be a a terpolymer comprising poly(caprolactone) or a poly(lactide-co-caprolactone). In one aspect, the terpolymer can be a terpolymer comprising poly(lactide-caprolactone), which, in various aspects, can be 95:5 poly(lactide-co-caprolactone), 85:15 poly(lactide-co-caprolactone), 75:25 poly(lactide-co-caprolactone), 65:35 poly(lactide-co- caprolactone), or 50:50 poly(lactide-co- caprolactone), where the ratios are mole ratios. In a further aspect, the terpolymer can comprise at least one of, or all of, lactide (D or L), glycolide, or caprolactone. Any two of lactide (D or L), glycolide, or caprolactone, when present, can be present in any desired ratio, with respect to the other. In one aspect, the terpolymer can be a poly(lactide-co-glycolide-co-caprolactone). In a further aspect, the terpolymer can be a poly(D-lactide-co-glycolide-co-caprolactone). The ratio of lactide to glycolide to caprolactone can be any desired ratio. In one aspect, the ratio of lactide to glycolide to caprolactone is 20:30:50. In one aspect, the terpolymer is a poly(D-lactide-co-glycolide-co-caprolactone) with at least one ester end cap, wherein the ratio of lactide to glycolide to caprolactone is 20:30:50, which has a viscosity of about 0.1 dL/g (abbreviated as 20:30:50 DLGCL 1E). In a further aspect, the terpolymer can be a terpolymer comprising an optionally endcapped polyethylene glycol. Examples of optionally endcapped polyethylene glycol include without limitation, polyethylene glycol (not endcapped), and mPEG, which is methoxypoly(ethylene glycol), among others. In one aspect, the terpolymer is a tacky terpolymer, such as, for example, a poly(D-lactide-co-glycolide-co-mPEG). In a further aspect, the terpolymer is a poly(D-lactide-co-glycolide-co-mPEG) wherein the ratio of lactide to glycolide is 50:50, and wherein the molecular weight of mPEG is about 2000 Daltons. Such a terpolymer can have an ester endcap and a viscosity of about 0.2 dL/g (abbreviated as 50:50 DLG mPEG 2000 2E).

In one aspect, the terpolymer comprises at least one of lactide, glycolide, caprolactone, optionally endcapped polyethylene glycol (PEG), or a combination thereof. In a further aspect, the terpolymer comprises a terpolymer of lactide, glycolide, and caprolactone residues, wherein the terpolymer comprises an end group that is a residue of an initiator and wherein the initiator is a non-crystalline primary or secondary alcohol. In a still further aspect, the terpolymer comprises at least one of poly(lactide-co-glycolide-co-optionally endcapped PEG), or poly(lactide-co-glycolide-co-caprolactone, or a combination thereof.

In one aspect, the terpolymers are those terpolymers disclosed in U.S. patent application Ser. No. 12/269135, filed Nov. 12, 2008, (U.S. Patent Publication No. 2009/0124535) which is incorporated herein by this reference for all of its teachings of terpolymers and is considered part of this disclosure.

It is understood that any combination of the aforementioned biodegradable terpolymers can be used, including, but not limited to, mixtures thereof, or blends thereof. Likewise, it is understood that when a residue of a biodegradable terpolymer is disclosed, any suitable terpolymer, mixture, or blend, that comprises the disclosed residue, is also considered disclosed. To that end, when multiple residues are individually disclosed (i.e., not in combination with another), it is understood that any combination of the individual residues can be used.

In one aspect, the terpolymer can be an adhering terpolymer that is capable of sticking to a contacting surface. In certain aspects, the terpolymer itself can be a tacky terpolymer, which functions as an adhesive to which a surface can directly adhere. Methods of making the above disclosed polymers tacky are known in the art. Addititives for example, can be added to provide a tacky terpolymer that can be adhesive. In one aspect, tacky polymers can be those that comprise a T_{g} of less than about room temperature, including those polymers disclosed above which have glass transition temperatures of less than about room temperature. In other aspects, the terpolymer can be a gel or gel-like, wax or wax-like, Vaseline® like, viscous, tacky, or a combination thereof.

As discussed above, the composition comprises a bioactive agent. The bioactive agent can be a releasable bioactive agent, i.e., a bioactive agent that can be released from the terpolymer. In certain aspects, the bioactive agent can be in or on the terpolymer.

Various forms of the bioactive agent can be used, which are capable of being released from terpolymer into adjacent tissues or fluids. To that end, a liquid or solid bioactive agent can be incorporated into the compositions described herein. The bioactive agents are at least very slightly water soluble, and preferably moderately water soluble. The bioactive agents can include salts of the active ingredient. As such, the bioactive agents can be acidic, basic, or amphoteric salts. They can be nonionic molecules, polar molecules, or molecular complexes capable of hydrogen bonding. The bioactive agent can be included in the compositions in the form of, for example, an uncharged molecule, a molecular complex, a salt, an ether, an ester, an amide, polymer drug conjugate, or other form to provide the effective biological or physiological activity.

Examples of bioactive agents that incorporated into systems herein include, but are not limited to, peptides, proteins such as hormones, enzymes, antibodies and the like, nucleic acids such as aptamers, iRNA, DNA , RNA, antisense nucleic acid or the like, antisense nucleic acid analogs or the like, low-molecular weight compounds, or high-molecular-weight compounds. Bioactive agents contemplated for use in the disclosed compositions include anabolic agents, antacids, anti-asthmatic agents, anti-cholesterolemic and anti-lipid agents, anti-coagulants, anti-convulsants, anti-diarrheals, anti-emetics, anti-infective agents including antibacterial and antimicrobial agents, anti-inflammatory agents, anti-manic agents, antimetabolite agents, anti-nauseants, anti-neoplastic agents, anti-obesity agents, anti-pyretic and analgesic agents, anti-spasmodic agents, anti-thrombotic agents, anti-tussive agents, anti-uricemic agents, anti-anginal agents, antihistamines, appetite suppressants, biologicals, cerebral dilators, coronary dilators, bronchiodilators, cytotoxic agents, decongestants, diuretics, diagnostic agents, erythropoietic agents, expectorants, gastrointestinal sedatives, hyperglycemic agents, hypnotics, hypoglycemic agents, immunomodulating agents, ion exchange resins, laxatives, mineral supplements, mucolytic agents, neuromuscular drugs, peripheral vasodilators, psychotropics, sedatives, stimulants, thyroid and anti-thyroid agents, tissue growth agents, uterine relaxants, vitamins, or antigenic materials.

Other bioactive agents include androgen inhibitors, polysaccharides, growth factors, hormones, anti-angiogenesis factors, dextromethorphan, dextromethorphan hydrobromide, noscapine, carbetapentane citrate, chlophedianol hydrochloride, chlorpheniramine maleate, phenindamine tartrate, pyrilamine maleate, doxylamine succinate, phenyltoloxamine citrate, phenylephrine hydrochloride, phenylpropanolamine hydrochloride, pseudoephedrine hydrochloride, ephedrine, codeine phosphate, codeine sulfate morphine, mineral supplements, cholestryramine, N-acetylprocainamide, acetaminophen, aspirin, ibuprofen, phenyl propanolamine hydrochloride, caffeine, guaifenesin, aluminum hydroxide, magnesium hydroxide, peptides, polypeptides, proteins, amino acids, hormones, interferons, cytokines, and vaccines.

Representative drugs that can be used as bioactive agents in the compositions include, but are not limited to, peptide drugs, protein drugs, desensitizing materials, antigens, anti-infective agents such as antibiotics, antimicrobial agents, antiviral, antibacterial, antiparasitic, antifungal substances and combination thereof, antiallergenics, androgenic steroids, decongestants, hypnotics, steroidal anti-inflammatory agents, anti-cholinergics, sympathomimetics, sedatives, miotics, psychic energizers, tranquilizers, vaccines, estrogens, progestational agents, humoral agents, prostaglandins, analgesics, antispasmodics, antimalarials, antihistamines, cardioactive agents, nonsteroidal anti-inflammatory agents, antiparkinsonian agents, antihypertensive agents, adrenergic blocking agents, nutritional agents, and the benzophenanthridine alkaloids. The agent can further be a substance capable of acting as a stimulant, sedative, hypnotic, analgesic, anticonvulsant, and the like.

The composition can comprise a large number of bioactive agents either singly or in combination. Other bioactive agents include but are not limited to analgesics such as acetaminophen, acetylsalicylic acid, and the like; anesthetics such as lidocaine, xylocaine, and the like; anorexics such as dexadrine, phendimetrazine tartrate, and the like; antiarthritics such as methylprednisolone, ibuprofen, and the like; antiasthmatics such as terbutaline sulfate, theophylline, ephedrine, and the like; antibiotics such as sulfisoxazole, penicillin G, ampicillin, cephalosporins, amikacin, gentamicin, tetracyclines, chloramphenicol, erythromycin, clindamycin, isoniazid, rifampin, and the like; antifungals such as amphotericin B, nystatin, ketoconazole, and the like; antivirals such as acyclovir, amantadine, and the like; anticancer agents such as cyclophosphamide, methotrexate, etretinate, and the like; anticoagulants such as heparin, warfarin, and the like; anticonvulsants such as phenytoin sodium, diazepam, and the like; antidepressants such as isocarboxazid, amoxapine, and the like;antihistamines such as diphenhydramine HCl, chlorpheniramine maleate, and the like; hormones such as insulin, progestins, estrogens, corticoids, glucocorticoids, androgens, and the like; tranquilizers such as thorazine, diazepam, chlorpromazine HCl, reserpine, chlordiazepoxide HCl, and the like; antispasmodics such as belladonna alkaloids, dicyclomine hydrochloride, and the like; vitamins and minerals such as essential amino acids, calcium, iron, potassium, zinc, vitamin B₁₂, and the like; cardiovascular agents such as prazosin HCl, nitroglycerin, propranolol HCl, hydralazine HCl, pancrelipase, succinic acid dehydrogenase, and the like; peptides and proteins such as LHRH, somatostatin, calcitonin, growth hormone, glucagon-like peptides, growth releasing factor, angiotensin, FSH, EGF, bone morphogenic protein (BMP), erythopoeitin (EPO), interferon, interleukin, collagen, fibrinogen, insulin, Factor VIII, Factor IX, Enbrel®, Rituxam®, Herceptin®, alpha-glucosidase, Cerazyme/Ceredose®, vasopressin, ACTH, human serum albumin, gamma globulin, structural proteins, blood product proteins, complex proteins, enzymes, antibodies, monoclonal antibodies, and the like; prostaglandins; nucleic acids; carbohydrates; fats; narcotics such as morphine, codeine, and the like, psychotherapeutics; anti-malarials, L-dopa, diuretics such as furosemide, spironolactone, and the like; antiulcer drugs such as rantidine HCl, cimetidine HCl, and the like.

The bioactive agent can also be an immunomodulator, including, for example, cytokines, interleukins, interferon, colony stimulating factor, tumor necrosis factor, and the like; allergens such as cat dander, birch pollen, house dust mite, grass pollen, and the like; antigens of bacterial organisms such as Streptococcus pneumoniae, Haemophilus influenzae, Staphylococcus aureus, Streptococcus pyrogenes, Corynebacterium diphteriae, Listeria monocytogenes, Bacillus anthracia, Clostridium tetani, Clostridium botulinum, Clostridium perfringens. Neisseria meningitides, Neisseria gonorrhoeae, Streptococcus mutans. Pseudomonas aeruginosa, Salmonella typhi, Haemophilus parainfluenzae, Bordetella pertussis, Francisella tularensis, Yersinia pestis, Vibrio cholerae, Legionella pneumophila, Mycobacterium tuberculosis, Mycobacterium leprae, Treponema pallidum, Leptspirosis interrogans, Borrelia burgddorferi, Campylobacter jejuni, and the like; antigens of such viruses as smallpox, influenza A and B, respiratory synctial, parainfluenza, measles, HIV, SARS, varicella-zoster, herpes simplex 1 and 2, cytomeglavirus, Epstein-Barr, rotavirus, rhinovirus, adenovirus, papillomavirus, poliovirus, mumps, rabies, rubella, coxsackieviruses, equine encephalitis, Japanese encephalitis, yellow fever, Rift Valley fever, lymphocytic choriomeningitis, hepatitis B, and the like; antigens of such fungal, protozoan, and parasitic organisms such as Cryptococcuc neoformans, Histoplasma capsulatum, Candida albicans, Candida tropicalis, Nocardia asteroids, Rickettsia ricketsii, Rickettsia typhi, Mycoplasma pneumoniae, Chlamyda psittaci, Chlamydia trachomatis, Plasmodium falciparum, Trypanasoma brucei, Entamoeba histolytica, Toxoplasma gondii, Trichomonas vaginalis, Schistosoma mansoni, and the like. These antigens may be in the form of whole killed organisms, peptides, proteins, glycoproteins, carbohydrates, or combinations thereof.

In a specific aspect, the bioactive agent comprises at least one of an antimicrobial, an antibiotic, a growth factor, a growth inhibitor, an immunomodulator, a steroid, or an anti-inflammatory, including any of those listed above.

In a further specific aspect, the bioactive agent comprises an antibiotic. The antibiotic can be, for example, one or more of Amikacin, Gentamicin, Kanamycin, Neomycin, Netilmicin, Streptomycin, Tobramycin, Paromomycin, Ansamycins, Geldanamycin, Herbimycin, Carbacephem, Loracarbef, Carbapenems, Ertapenem, Doripenem, Imipenem/Cilastatin, Meropenem, Cephalosporins (First generation), Cefadroxil, Cefazolin, Cefalotin or Cefalothin, Cefalexin, Cephalosporins (Second generation), Cefaclor, Cefamandole, Cefoxitin, Cefprozil, Cefuroxime, Cephalosporins (Third generation), Cefixime, Cefdinir, Cefditoren, Cefoperazone, Cefotaxime, Cefpodoxime, Ceftazidime, Ceftibuten, Ceftizoxime, Ceftriaxone, Cephalosporins (Fourth generation), Cefepime, Cephalosporins (Fifth generation), Ceftobiprole, Glycopeptides, Teicoplanin, Vancomycin, Macrolides, Azithromycin, Clarithromycin, Dirithromycin, Erythromycin, Roxithromycin, Troleandomycin, Telithromycin, Spectinomycin, Monobactams, Aztreonam, Penicillins, Amoxicillin, Ampicillin, Azlocillin, Carbenicillin, Cloxacillin, Dicloxacillin, Flucloxacillin, Mezlocillin, Meticillin, Nafcillin, Oxacillin, Penicillin, Piperacillin, Ticarcillin, Polypeptides, Bacitracin, Colistin, Polymyxin B, Quinolones, Ciprofloxacin, Enoxacin, Gatifloxacin, Levofloxacin, Lomefloxacin, Moxifloxacin, Norfloxacin, Ofloxacin, Trovafloxacin, Sulfonamides, Mafenide, Prontosil (archaic), Sulfacetamide, Sulfamethizole, Sulfanilimide (archaic), Sulfasalazine, Sulfisoxazole, Trimethoprim, Trimethoprim-Sulfamethoxazole (Co-trimoxazole) (TMP-SMX), Tetracyclines, including Demeclocycline, Doxycycline, Minocycline, Oxytetracycline, Tetracycline, and others; Arsphenamine, Chloramphenicol, Clindamycin, Lincomycin, Ethambutol, Fosfomycin, Fusidic acid, Furazolidone, Isoniazid, Linezolid, Metronidazole, Mupirocin, Nitrofurantoin, Platensimycin, Pyrazinamide, Quinupristin/Dalfopristin, Rifampicin (Rifampin in U.S.), Tinidazole, or a combination thereof. In one aspect, the bioactive agent can be a combination of Rifampicin (Rifampin in U.S.) and Minocycline.

It is contemplated that other components such as, for example, excipients, pharmaceutically carriers or adjuvants, microparticles, and the like, can be combined with the terpolymer and/or the bioactive agent. Thus, in certain aspects, the bioactive agent can be present as a component in a pharmaceutical composition. Pharmaceutical compositions can be conveniently prepared in a desired dosage form, including, for example, a unit dosage form or controlled release dosage form, and prepared by any of the methods well known in the art of pharmacy. In general, pharmaceutical compositions are prepared by uniformly and intimately bringing the bioactive agent into association with a liquid carrier or a finely divided solid carrier, or both. The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen. Other pharmaceutically acceptable carriers or components that can be mixed with the bioactive agent can include, for example, a fatty acid, a sugar, a salt, a water-soluble polymer such as polyethylene glycol, a protein, polysacharride, or carboxmethyl cellulose, a surfactant, a plasticizer, a high- or low-molecular-weight porosigen such as polymer or a salt or sugar, or a hydrophobic low-molecular-weight compound such as cholesterol or a wax.

In certain aspects, the terpolymer and bioactive agent are combined or admixed to form a blend or admixture. Admixing methods can be performed using techniques known in the art. For example, the terpolymer and bioactive agent can be dry blended (i.e., mixing of particulates of the terpolymer and the agent) using, for example, a Patterson-Kelley V-blender, or granulated prior to processing.

In one aspect, the processing of the admixture can be performed under conditions such that the agent is intimately mixed or dispersed throughout the terpolymer. Alternatively, the processing of the admixture can be performed under conditions such that the agent is localized on or in only a portion or portions of the terpolymer. To that end, the terpolymer can include areas that are rich in bioactive agent, and areas that are not as rich. The admixture can be processed by a variety of techniques, such as, for example, melt extruding, injection molding, compression molding, or roller compacting the admixture into a desired shape or structure. Other suitable pharmaceutical carriers include without limitation microparticles. The term “microparticle” is used herein to refer generally to a variety of substantially structures having sizes from about 10 nm to 2000 microns (2 millimeters) and includes microcapsule, microsphere, nanoparticle, nanocapsule, nanosphere as well as particles, in general, that are less than about 2000 microns (2 millimeters). The microparticle can contain and effect the release of the bioactive agent from the terpolymer.

The microparticle can be comprised of any of those polymers mentioned above or any polymer used in the microparticle art. In general, the above mentioned polymers can be cross-linked to a certain level, which thereby can form a microparticle of the polymer, as is known in the art. When a microparticle is present in the polymer matrix, the microparticle can be the same or different as the polymer comprising the bulk of the polymer matrix. The polymer matrix can comprise any desired amount of microparticles, including, for example, from about 1 weight % to about 95 weight %, including 5, 10, 20, 30, 40, 50, 60, 70, 80, and 90 weight %, relative to the weight of the total polymer matrix. The microparticle can be combined with the polymer matrix through known methods.

In one aspect, the disclosed microparticles can have an average or mean particle size of from about 20 microns to about 125 microns. In one embodiment the range of mean particle size is from about 40 microns to about 90 microns. In another embodiment the range of mean particle sizes is from about 50 microns to about 80 microns. Particle size distributions are measured by laser diffraction techniques known to those of skill in the art.

In a further aspect, the bioactive agent can be encapsulated, microencapsulated, or otherwise contained within a microparticle. The microparticle can modulate the release of the bioactive agent. The microparticle can comprise any desired amount of the bioactive agent. For example, the microparticle can comprise 1%, 5%, 10%, 20%, 30%, 40%, 50%, 60%, 70%, 80%, 90%, 95% by weight bioactive agent, relative to the weight of the microparticle, including any range between the disclosed percentages.

The microparticles can be made using methods known in the art, including, for example, those methods disclosed in U.S. Patent Publication No. 2007/0190154, published Aug. 16, 2007, and U.S. Pat. No. 5,407,609 to Tice et al., both of which are incorporated herein in their entirety by this reference for teachings of microparticle preparation methods. As will be apparent, depending upon processing conditions, the terpolymer used as a starting material in the admixing step may or may not be the same terpolymer present in the final composition. For example, the terpolymer during processing may undergo polymerization or depolymerization reactions, which ultimately can produce a different terpolymer that was used prior to processing. Thus, the term “terpolymer” as used herein covers the polymers used as starting materials as well as the final terpolymer present in the device produced by the methods described herein.

In one aspect, the terpolymer can be stored separately from the bioactive agent, and the composition can be formulated near or at the time of use. Such aspects allow for the drug stability to remain independent, and/or free from contamination or alteration due to the presence of the terpolymer. It should also be appreciated that many of those biodegradable, tacky terpolymers are well characterized and generally accepted by the FDA for parenteral use.

Also disclosed are kits comprising the compositions. A kit can have various compositions that can be same or different depending on their intended use, all packaged together in one unit.

In a further aspect, a kit comprises 1) an adhering biocompatible terpolymer; and 2) a bioactive agent; wherein the terpolymer and bioactive agent are separated. In one aspect, the terpolymer is present in a first container, and the bioactive agent is present in a second container. In a further aspect, the terpolymer is present in a first dispensing system, and the bioactive agent is present in a second dispensing system. To that end, the first dispensing system can dispense the terpolymer, while the second dispensing system can dispense the bioactive agent, such that a mixture is formed that comprises the terpolymer and the bioactive agent. Such a process can be conveniently carried out, in various aspects, using a syringe as the first and/or second dispensing system.

The compositions, as discussed above in various aspects, can be provided by admixing the adhering biocompatible terpolymer and the bioactive agent to form a composition. In one aspect, the terpolymer and the bioactive agent can be stored separately until close to, or at, the time of use. For example, the terpolymer and the bioactive agent can be stored separately in a disclosed kit.

In one aspect, a method for formulating a bioactive composition comprises mixing the adhering biocompatible terpolymer and the bioactive agent, thereby forming the bioactive composition. The mixing step can be performed at any time. In one aspect, the mixing step is performed close to, or at, the time of use, or close to, or at, the time of implant surgery, including without limitation on the same day (i.e., within 24 hours) of the implant surgery, including, for example, within 23 hours, 20 hours, 15 hours, 10 hours, 5 hours, 3 hours, 2 hours, 1 hour, 30 minutes, 15 minutes, 10 minutes, 5 minutes, 2 minutes, 30 seconds, or during the implant surgery itself.

Also disclosed are implant devices comprising the compositions. The term “device” is any formulation or article that is greater than 1 mm in length in at least one dimension of the device. The device can comprise a disclosed composition. In a further aspect, the device has one dimension that is from 1 mm to 50 mm, 1.2 mm to 45 mm, 1.4 mm to 42 mm, 1.6 mm to 40 mm, 1.8 mm to 38 mm, or 2.0 mm to 36 mm, 5.0 mm to 33 mm, or 10 mm to 30 mm. In a further aspect, the device has one dimension that is greater than 3 cm, even up to or greater than 10 cm, 20 cm, or even 30 cm.

In one aspect, the implant device comprises at least a first implant device surface comprising a composition on at least a portion thereof, wherein if the composition has a length dimension substantially larger than a width dimension and a width dimension substantially larger than a thickness dimension, then the width dimension is less than about 2 mm.

The implant device can comprise any shape, such as a rod, a fiber, a cylinder, a bead, a ribbon, a disc, a wafer, a free-formed shaped solid, or a variety of other shaped solids. The device can have any regular or irregular shape and can have any cross section like circular, rectangular, triangular, oval, and the like. In a further aspect, the device comprises a cylindrical shape, such as a typical shape of an implantable pump.

The implant can be comprised of any suitable material, such as a metal (e.g., titanium), metal composite, organic material, polymeric, or even ceramic material. The surface of the implant can be any shaped surface, and may have a porous, beaded or meshed ingrowth surface, as can be present in certain implants.

The implant device can be any type of medical implant. The implant devices can include, for example, implants for drug delivery, including drug delivery pumps; orthopedic implants, including spinal implants, implants for osseointegration or bone repair; medical stents, including stents with inherent drug delivery capability; prosthetic implants, including breast implants, muscle implants, and the like; dental implants; ear implants, including cochlear implants and hearing devices; cardiac implants including pacemakers, catheters, etc.; space filling implants; bioelectric implants; neural implants; internal organ implants, including dialysis grafts; defribrillators; monitoring devices; recording devices; stimulators, including deep brain stimulators, nerve stimulators, bladder stimulators, and diaphragm stimulators; implantable identification devices and information chips; artificial organs; drug administering devices; implantable sensors/biosensors; screws; tubes; rods; plates; or artificial joints.

In a further aspect, the implant device can be at least one of a pump, pacemaker, defribrillator, or stimulator, including deep brain stimulators, nerve stimulators, bladder stimulators, and diaphragm stimulators.

Other implant devices that may benefit when used with the disclosed compositions include those with one or more active surfaces, e.g., a surface that enhances a connection between a tissue or fluid and the implant device, or a surface that allows for or enhances wound healing. To that end, the disclosed compositions can be effective when applied to only a portion of the implant device, allowing for any active surface to remain exposed and functional when the implant device is implanted in a subject.

As discussed above, it can be desirable to deliver a bioactive agent at or near the tissue adjacent an implant site. The bioactive agent can help prevent some of the problems associated with implants, such as infection, or enhance the function of the implant itself. It can also be desirable to avoid pre-manufacturing an implant device with bioactive agent releasing capability, as discussed above. It should be appreciated that the compositions, methods, and kits disclosed herein can allow for a point of use application of a composition onto the surface of an implant device, thus obviating the need to pre-manufacture implant devices having bioactive agent releasing capability.

In one aspect, a composition or coating (e.g., a coating wherein the terpolymer and bioactive agent are separated) can be applied to an implant device surface close to or during the time of use. For example, a composition can be applied to an implant device by applying (e.g. rubbing, brushing, smearing, dispensing from a dispensing system or kit, etc.) the composition or coating onto the surface of the implant device, substantially close to the time when the implant device is implanted in a subject. In one aspect, the composition or coating can be applied to an implant device in an operating suite, for example, by a physician or nurse.

In a further aspect, an implant device can be coated with a coating comprising the 1) adhering biocompatible terpolymer and 2) the bioactive agent of a disclosed kit onto a surface of the implant device. In one aspect, the 1) adhering biocompatible terpolymer and 2) the bioactive agent are mixed prior to, at, or after the coating is applied to the surface of the implant device. Thus, the term “coating” includes without limitation at least two coatings, wherein the at least two coatings are separated from each other, on the device surface. For example, a first region of the device surface can comprise the terpolymer, and a second region of the device surface can comprise the bioactive agent, which is separated from the terpolymer. Such a coating can be formed into a disclosed composition by mixing the terpolymer and the bioactive agent, when they are present on the device surface. When present separately on a device surface, the terpolymer and the bioactive agent can be mixed using known methods, for example, by smearing the two coatings together with an applicator or q-tip. In a further aspect, the terpolymer and the bioactive agent are mixed. In a still further aspect, the terpolymer and the bioactive agent are first mixed and then applied, as a composition, to the implant device surface.

When the terpolymer and the bioactive agent are stored or kept separately from each other until the time of use, the terpolymer and bioactive agent can be mixed at any desired time. In one aspect, the adhering biocompatible terpolymer and the bioactive agent are mixed prior to applying the coating to the device surface. Thus, in this aspect, a composition comprising the terpolymer and the bioactive agent is first formed, prior to applying the coating to the device surface. In a further aspect, as discussed above, the terpolymer and the bioactive agent are mixed after each are individually and separately present on the device surface. In a still further aspect, the terpolymer and the bioactive agent are mixed at the same time, or a time close to the time, that each are individually being applied to the device surface.

The compositions and coatings can be applied to the surface of the implant device prior to or after the time when the implant device is implanted in the subject. In one aspect, the implant device comprising the composition and/or coatings can be implanted into the subject. In a further aspect, the implant device can be implanted into the subject, and then the composition and/or coating can be applied to the surface of the implant device. When implanting smaller implants, it may be beneficial to first apply the composition and/or coating to the implant device surface.

In one aspect, the composition and/or coating can be applied to the surface of the implant device on the same day (i.e., within 24 hours) of the implant surgery, including, for example, within 23 hours, 20 hours, 15 hours, 10 hours, 5 hours, 3 hours, 2 hours, 1 hour, 30 minutes, 15 minutes, 10 minutes, 5 minutes, 2 minutes, 30 seconds, or during the implant surgery itself.

If desired, the composition and/or coating itself, with or without an implant device, can be implanted onto or in a tissue or fluid of a subject. In one aspect, the composition and/or coating can be implanted onto or in a tissue or fluid that is near or adjacent to an implant site, i.e., a site where an implant device has been implanted, or near or adjacent to a desired implant site.

Typically, before applying the composition and/or coating to the implant device, the implant device surface can be cleaned or treated to remove any surface contaminants and to promote good adhesion of the terpolymer. For example, the composition, coating and/or the implant device can be sterilized. The composition and/or coating, or implant device comprising the composition and/or coating can then be implanted into the subject using known surgical techniques. In certain aspects, it can be desirable to store the compositions, components thereof, or kits comprising the compositions, or components thereof, in a sterilized container or package. In one aspect, the kit can comprise a sterilized package of the compositions, or components thereof, as discussed above.

In one aspect, the disclosed methods can be used with compositions comprising a releasable bioactive agent. In a further aspect, the methods can be used with compositions comprising an adhering biocompatible terpolymer and a bioactive agent. The composition can be applied to the surface of the implant, or to the tissue or fluid of the subject, by contacting the surface of the implant with the terpolymer composition, or component thereof.

The implant device can be implanted in any desired subject. The subject can be a vertebrate, such as a mammal, a fish, a bird, a reptile, or an amphibian. The subject of the herein disclosed methods can be, for example, a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

Efforts have been made to ensure accuracy with respect to numbers (e.g., amounts, temperature, pH, etc.) but some errors and deviations should be accounted for. Unless indicated otherwise, parts are parts by weight, temperature is in ° C. or is at ambient temperature, and pressure is at or near atmospheric. There are numerous variations and combinations of conditions, e.g., component concentrations, temperatures, pressures, and other reaction ranges and conditions that can be used to optimize the product purity and yield obtained from the described process. Only reasonable and routine experimentation will be required to optimize such process conditions.

### Example 1

### (Prophetic)

In a first example, a terpolymer composition is provided. A first syringe contains a viscous terpolymer such as 20:30:50 DLGCL 1E. A second syringe contains the bioactive agent, which in this case is an antibiotic, dissolved or suspended in a polymer compatible solvent (DMSO, NMP, etc). Prior to applying the composition to an implant device surface, the two syringes can be connected, and the components can be mixed by pushing the syringe plungers back and forth. The bioactive agent content can range from 0.1 wt % up to approximately 60 wt %, or greater, depending on the desired dose. The resulting viscous solution/suspension can be directly applied to the implant device surface with the syringe. A controlled release of the active agent can be achieved with a duration ranging from hours to days, as discuused above.

### Example 2

### (Prophetic)

In a second example, another terpolymer composition is provided. A first syringe contains a viscous terpolymer such as 20:30:50 DLGCL 1E. A second syringe contains the bioactive agent in the form of a dry powder. The two syringes are connected and the active agent is suspended in the terpolymer by pushing the syringe plungers back and forth. The resulting viscous suspension can be directly applied to an implant device surface with the syringe. Similarly, the drug content can range from 0.1 wt % up to approximately 60 wt %, or greater as needed to control dose and release rate.

### Example 3

### (Prophetic)

In a third example, a single syringe method for providing a terpolymer composition is disclosed. The terpolymer, active agent, and cosolvent (if desired or necessary) can be combined together and packaged in a single syringe. The drug content can range from 0.1 wt % up to about 60 wt %, or greater, depending on the desired dose. The resulting viscous solution/suspension can be directly applied to the surface of an implant device with the syringe. A controlled release of the active agent can be achieved with a duration ranging from hours to days.

### Example 4

### (Prophetic)

In a fourth example, a single packing system comprising a terpolymer composition is provided. A single syringe or other suitable packaging device contains a tacky terpolymer such as 50:50 DLG mPEG 2000 2E. A second syringe or packaging device contains the bioactive agent in the form of a dry powder or film. The tacky terpolymer can be applied and spread onto the surface of the implant device, thus providing an adhesive surface to which the bioactive agent formulation can be applied. The bioactive agent formulation can then be applied to the tacky terpolymer surface. A controlled release of the bioactive agent can be achieved with a duration ranging from hours to days.

### Example 5

### (Prophetic)

In a fifth example, another terpolymer composition is provided. A single syringe system or other suitable packaging device comprises the terpolymer such as 50:50 DLG mPEG 2K, the bioactive agent, and cosolvent (if desired or necessary). The bioactive agent content can range from 0.1 wt % up to about 60 wt %, or greater, depending on the desired dose. The resulting viscous solution/suspension can be directly applied to an implant device surface with the syringe and spread onto the surface of the device. A controlled release of the active agent can be achieved with a duration ranging from hours to days.

Various modifications and variations can be made to the compounds, compositions, kits, articles, devices, compositions, and methods described herein. Other aspects of the the compounds, compositions, kits, articles, devices, compositions, and methods described herein will be apparent from consideration of the specification and practice of the the compounds, compositions, kits, articles, devices, compositions, and methods disclosed herein. It is intended that the specification and examples be considered as exemplary.

1-6. (canceled)

7. A kit comprising an adhering biocompatible terpolymer and a bioactive agent.

8. The kit of claim 7, wherein the terpolymer and bioactive agent are separated.

9. The kit of claim 7, wherein the terpolymer is present in a first container, and the bioactive agent is present in a second container.

10. The kit of claim 7, wherein the terpolymer is present in a first dispensing system, and the bioactive agent is present in a second dispensing system.

11. The kit of claim 10, wherein at least one of the first dispensing system or the second dispensing system comprises a syringe.

12. The kit of claim 7, wherein the terpolymer comprises at least one of lactide, glycolide, caprolactone, optionally endcapped polyethylene glycol (PEG), or a combination thereof.

13. The kit of claim 7, wherein the terpolymer comprises lactide, glycolide, and caprolactone residues, and wherein the terpolymer comprises an end group that is a residue of an initiator that is a non-crystalline primary or secondary alcohol.

14. The kit of claim 7, wherein the bioactive agent is encapsulated within a microparticle.

15. The kit of claim 7, wherein the bioactive agent comprises at least one of an antibiotic, a growth factor, an immunomodulator, a steroid, or an anti-inflammatory.

16-18. (canceled)
